(19) United States
(12) Patent Application Publication
(10) Pub. No.: US 2010/0087708 A1
(43) Pub. Date: Apr. 8, 2010

(54) SEPARABLE-TYPE ENDOSCOPE IMAGING SYSTEM

(75) Inventors: WEN-SHANG CHEN, Taoyuan County (TW); KIN-CHEONG CHAN, Taoyuan County (TW); CHIH-CHUN CHAN, Miaoli County (TW)

Correspondence Address: WPAT, PC;INTELLECTUAL PROPERTY ATTORNEYS, 2030 MAIN STREET, SUITE 1300, IRVINE, CA, 92614, US

(73) Assignee: Medical Intubation Technology Corporation, Taoyuan Hsien (TW)

(21) Appl. No.: 12/489,297

(22) Filed: Jun. 22, 2009

(30) Foreign Application Priority Data
Oct. 7, 2008 (TW) 097138591

(51) Int. Cl. A61B 1/04

(52) U.S. Cl. 600/112

(57) ABSTRACT

A separable-type endoscope imaging system includes a first body portion, a flexible hollow detection tube, and a second body portion. The first body portion includes an output terminal and a wireless transmitter unit. The flexible hollow detection tube is located at one end of the first body portion and internally provided with a lens assembly for generating an image signal. The second body portion includes an input terminal, a wireless receiver unit, and a display unit. When the output terminal is electrically connected to the input terminal, the image signal is transmitted via the output terminal and the input terminal to the display unit for displaying. On the other hand, when the output terminal is not electrically connected to the input terminal, the image signal is wirelessly transmitted from the wireless transmitter unit to the wireless receiver unit for displaying on the display unit.

### FIELD OF THE INVENTION

The present invention relates to an endoscope imaging system, and more particularly to a separable-type endoscope imaging system, with which an image signal can be selectively wire transmitted or wirelessly transmitted to a display unit for displaying.

### BACKGROUND OF THE INVENTION

In recent years, endoscope imaging devices for use in endoscopy have been gradually upgraded and widely applied in different fields. However, a conventional wired endoscope imaging device is largely restricted by the limited signal transmission distance thereof and could not be controlled from a remote position. Moreover, the flexible tube for the wired endoscope imaging device can not be extended to a distant place or through a complicated landform or a structurally complicated object. As a result, the application range of the wired endoscope imaging system is largely limited. To overcome this problem, a wireless endoscope imaging device has been developed.

The conventional wireless endoscope imaging device usually includes a first body portion being provided with a wireless transmitter unit, a flexible tube, an imaging unit, and a light source unit; and a second body portion being provided with a wireless receiver unit and a display screen. Light projected from the light source unit is reflected from an object being inspected to the imaging unit, causing the imaging unit to generate an image signal, which is transmitted from the wireless transmitter unit to the wireless receiver unit and be displayed on the display screen.

The conventional wireless endoscope imaging device is advantageous in that it has a long transmission distance not limited by the length of the flexible tube. Therefore, more than one user can watch the display screen at a remote place.

However, the conventional wireless endoscope imaging device has a critical problem, i.e., the wirelessly transmitted image signal is subject to interference by other nearby strong wireless signals, especially when these nearby wireless signals use the same radio frequency as that of the wireless transmitter unit of the wireless endoscope imaging device. On the other hand, when the conventional wireless endoscope imaging device is used in some working sites that do not allow good transmission of a wireless signal, such as in an elevator, the second body portion of the wireless endoscope imaging device would not be able to receive the image signal wirelessly transmitted from the first body portion for displaying on the display screen of the second body portion. Moreover, a user is prevented from handling other works while using the wireless endoscope imaging device because the user has to hold the first and the second body portion with two hands.

### SUMMARY OF THE INVENTION

A primary object of the present invention is to provide a separable-type endoscope imaging system that allows an image signal to be wirelessly transmitted from a wireless transmitter unit on a first body portion to a wireless receiver unit on a second body portion for displaying on a display unit on the second body portion.

Another object of the present invention is to provide a separable-type endoscope imaging system that allows an image signal to be wire transmitted from an output terminal on a first body portion to an input terminal on a second body portion for displaying on the display unit on the second body portion.

To achieve the above and other objects, the separable-type endoscope imaging system according to the present invention includes a first body portion, a flexible hollow detection tube, and a second body portion. The first body portion includes an output terminal and a wireless transmitter unit. The flexible hollow detection tube is located at one end of the first body portion and is internally provided with a lens assembly for generating an image signal. The second body portion includes an input terminal, a wireless receiver unit, and a display unit. When the output terminal is electrically connected to the input terminal, the image signal is transmitted from the first body portion to the second body portion via the output terminal and the input terminal to be displayed on the display unit on the second body portion. On the other hand, when the output terminal is not electrically connected to the input terminal, the image signal is wirelessly transmitted from the wireless transmitter unit to the wireless receiver unit to be displayed on the display unit.

With the above arrangements, the separable-type endoscope imaging system of the present invention has the following advantages:
(1) The separable-type endoscope imaging system enables the image signal to be wire transmitted without being interfered by nearby wireless signals.
(2) When it is used to wire transmit the image signal, the separable-type endoscope imaging system can be placed on a plane without the need of being held by a user, so that the user can handle other works with two hands.
(3) The flexible hollow detection tube of the separable-type endoscope imaging system can be adjusted in angle, allowing a user to view the display unit from the best view angle without the need of bending or kneeing on the ground.
(4) When the separable-type endoscope imaging system is used to wirelessly transmit the image signal, the user can conveniently view the display unit at a remote place without being limited to a short signal transmission range as in the case of wire transmission.

In brief, the separable-type endoscope imaging system of the present invention has simplified structure and enables effective transmission of the image signal.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a perspective view showing a first body portion and a flexible hollow detection tube of a separable-type endoscope imaging system according to a preferred embodiment of the present invention;

FIG. 2 is a front perspective view of a second body portion of the separable-type endoscope imaging system of the present invention;

FIG. 3 is a rear perspective view of the second body portion of the separable-type endoscope imaging system of the present invention;

FIG. 4 shows the manner of assembling the first body portion and the flexible hollow detection tube to the second body portion of the separable-type endoscope imaging system of the present invention;

FIG. 5 is a block diagram showing wire transmission of an image signal from the first body portion to the second body portion of the separable-type endoscope imaging system of the present invention; and

FIG. 6 is a block diagram showing wireless transmission of an image signal from the first body portion to the second body portion of the separable-type endoscope imaging system of the present invention.

### DETAILED DESCRIPTION OF THE PREFERRED EMBODIMENTS

It is known that an endoscope device mainly includes a flexible hollow detection tube, a lens assembly, and a light source. The endoscope device can be connected to a display, so that the internal structure of, for example, a body organ, a traffic means, components of an electronic device, a fissure on a building, etc. being inspected can be shown on the display, via which a doctor, an architect, an electronic engineer, a mechanism engineer or technician, etc. can examine, diagnose, or otherwise find out any problem with the object being inspected. So long as a damaged body organ or mechanical part can be reached from an external environment via a natural opening, the endoscope device can be utilized to examine such damaged body organ or mechanical part. For example, an upper gastrointestinal endoscope device can be positioned in a patient's gullet, stomach, and duodenum via the patient's mouth, a colonoscope can be positioned in a patient's colon via the patient's anus, and an endoscope device for industrial purpose can be extended through an exhaust port for checking turbine blades. In the event there is not a natural opening, a man-made opening can be built by surgery in the case of a patient, or loosening some screws in the case of a machine. For example, a small surgical incision must first be made on the patient's abdomen to enable a laparoscopy, the patient's skin around a damaged joint must first be cut open for arthroscopy, and a housing of an engine must be first removed for inspection with an endoscope. Therefore, the endoscope device has wide applications in various fields, particularly in medical and industrial fields.

The present invention directs to a separable-type endoscope imaging system, which will now be described with a preferred embodiment thereof. For the purpose of easy to understand, elements that are the same in the preferred embodiment are denoted by the same reference numerals.

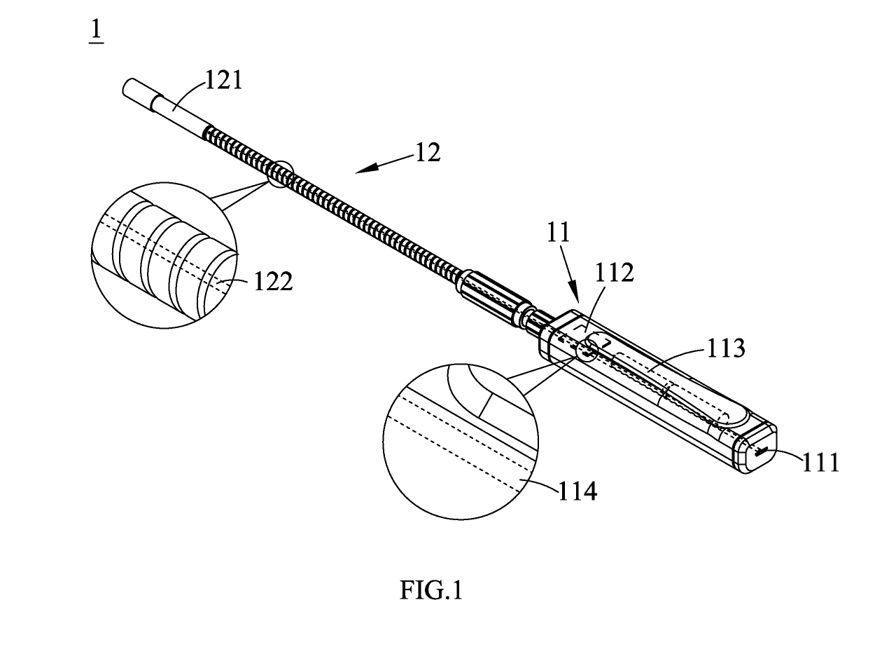

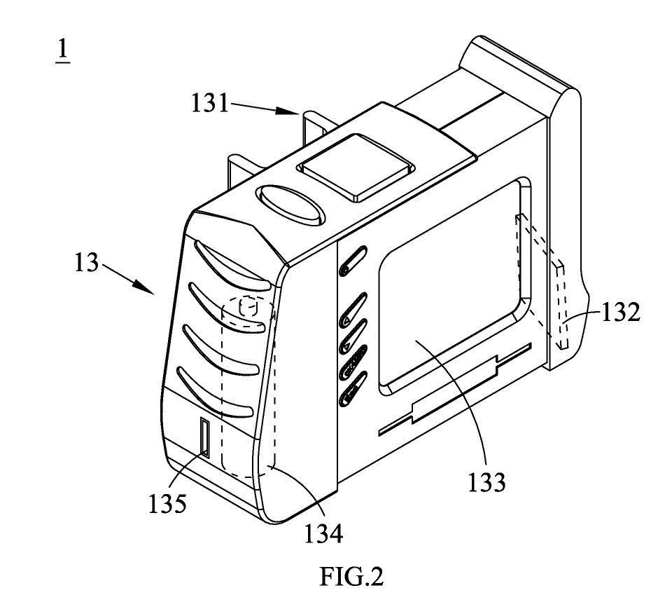

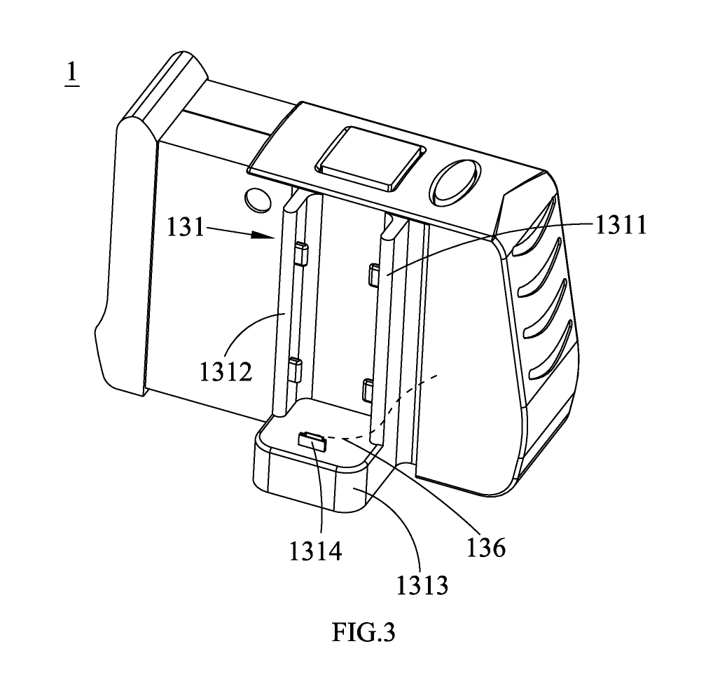

Please refer to FIG. 1 that is a perspective view showing a first body portion 11 and a flexible hollow detection tube 12 of a separable-type endoscope imaging system 1 according to a preferred embodiment of the present invention, and to FIGS. 2 and 3, which are front and rear perspective views, respectively, of a second body portion 12 of the separable-type endoscope imaging system 1. As shown, the first body portion 11, the flexible hollow detection tube 12, and the second body portion 13 form three major parts of the separable-type endoscope imaging system 1.

The first body portion 11 is configured for easy holding and handling by a user. As shown, on the first body portion 11, there are provided an output terminal 111, a wireless transmitter unit 112, a first power supply unit 113, and a first physical line 114. In the illustrated preferred embodiment, the output terminal 111, the wireless transmitter unit 112, the first power supply unit 113, and the first physical line 114 are all arranged inside the first body portion 11. The output terminal 111 is located at a bottom or rear end of the first body portion 11 with a first end connected to a first end of the first physical line 114 and a second end electrically connectable to an input terminal 1314 on the second body portion 13. The wireless transmitter unit 112 enables wireless transmission of an image signal (not shown). The first power supply unit 113 can be, for example, a battery for supplying power to the first body portion 11 and the second body portion 13. The first physical line 114 enables wire transmission of an image signal via the line.

The flexible hollow detection tube 12 is located at and separably connected to one end of the first body portion 11 to enable convenient replacement of the tube with a new one. In the illustrated preferred embodiment, the flexible hollow detection tube 12 is a flexible tube having a lens assembly 121 and a second physical line 122 provided therein. The second physical line 122 has a first end connected to the lens assembly 121 and a second end to a second end of the first physical line 114. With the lens assembly 121, an image signal (not shown) can be generated. Via the second physical line 122, wire transmission of the generated image signal to the first solid wire 114 or the wireless transmitter unit 112 can be effectuated.

On the second body portion 13, there are provided a fixing seat 131, a wireless receiver unit 132, a display unit 133, a second power supply unit 134, a display jack 135, and a third physical line 136. In the illustrated preferred embodiment, the wireless receiver unit 132, the second power supply unit 134, and the third physical line 136 are arranged inside the second body portion 13 while the fixing seat 131 is externally arranged at a rear side of the second body portion 13. The fixing seat 131 is defined by a left section 1311, a right section 1312, and a bottom section 1313. The input terminal 1314 is provided on the bottom section 1313 and electrically connectable to the output terminal 111 on the first body portion 11. The wireless receiver unit 132 is adapted to wirelessly receive an image signal.

The display unit 133 is located at a front side of the second body portion 13 for displaying images. The second power supply unit 134 can be a battery for supplying power to the first body portion 11 and the second body portion 13. The display jack 135 is located at a lateral side on the second body portion 13 for an external display unit (not shown) to connect thereto. When an external display unit is connected to the display jack 135, the image signal can be transmitted via the display jack 135 to the external display unit. Alternatively, when an external display unit is connected to the display jack 135, the image signal can be transmitted to the display unit 133 while also transmitted via the display jack 135 to the external display unit. The third physical line 136 enables wire transmission of an image signal.

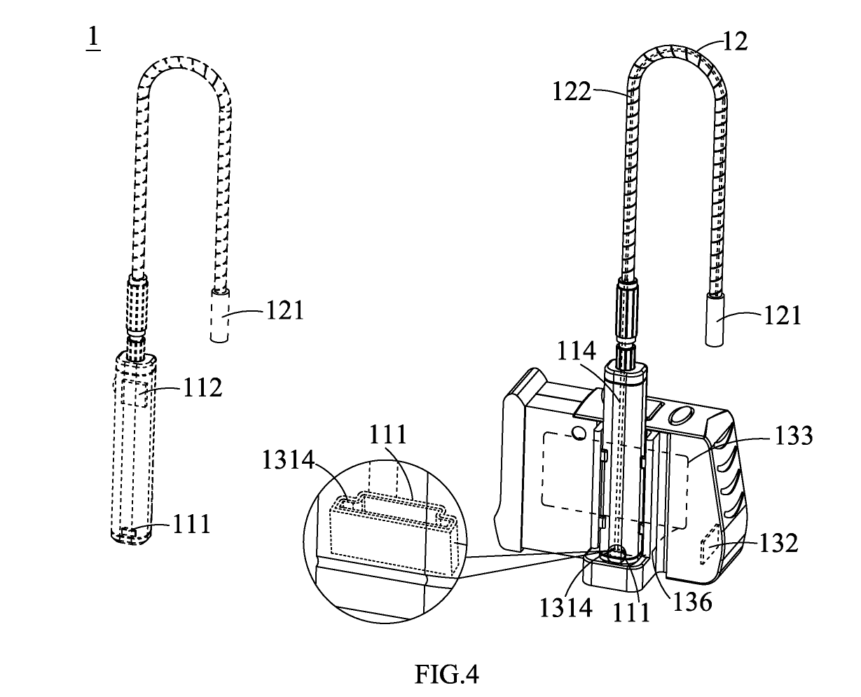

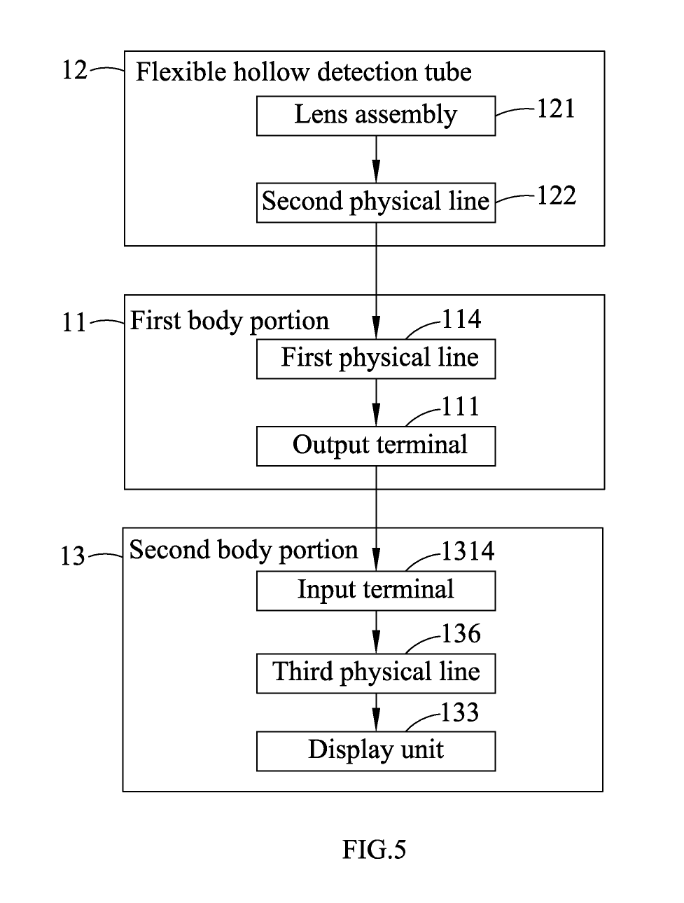

FIG. 4 shows the assembling of the first body portion 11 and the hollow detection tube 12 to the second body portion 13 of the separable-type endoscope imaging system 1, and FIG. 5 is a block diagram showing wire transmission of an image signal from the first body portion 11 to the second body portion 13. Please refer to FIGS. 4 and 5 at the same time. When the output terminal 111 on the first body portion 11 is electrically connected to the input terminal 1314 on the second body portion 13, the image signal (not shown) generated by the lens assembly 121 is transmitted to the display unit 133 on the second body portion 13 via the second physical line 122 in the flexible hollow detection tube 12, the first physical line 114 and the output terminal 111 on the first body portion 11, and the input terminal 1314 and the third physical line 136 on the second body portion 13. With the wire transmission provided by the separable-type endoscope imaging system 1, the image signal can be transmitted without being interfered by nearby wireless signals. Also, with the above arrangements, the separable-type endoscope imaging system 1 can be placed on a plane without the need of being held by a user. Thus, the user can handle other works with two hands. Meanwhile, the flexible hollow detection tube 12 can be adjusted to a desired angle for the user to view the display unit 133 from a best view angle without the need of bending or kneeing on the ground.

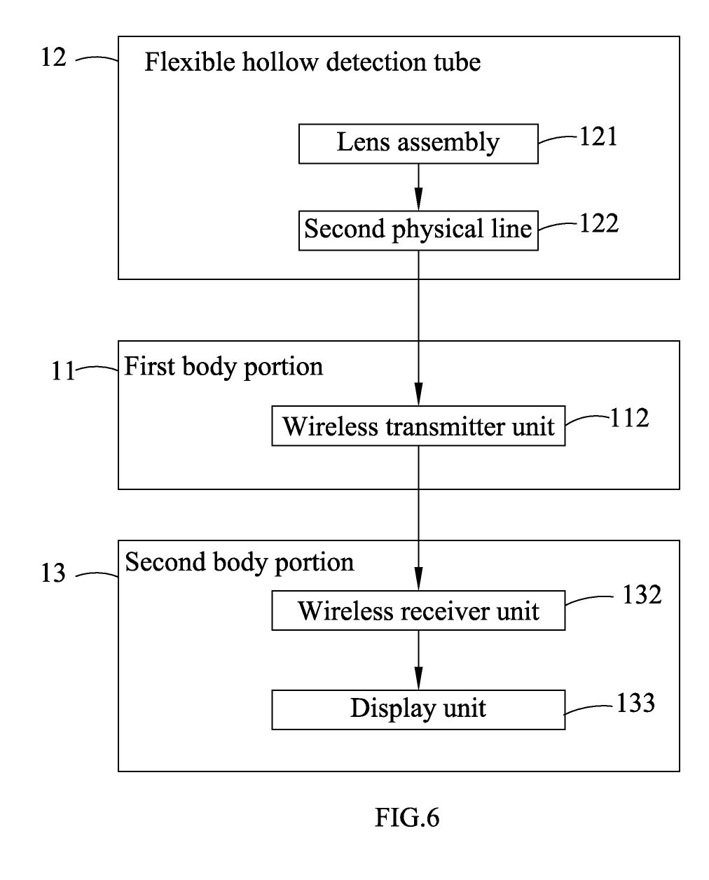

Please now refer to FIG. 6, which is a block diagram showing wireless transmission of an image signal from the first body portion 11 to the second body portion 13 of the separable-type endoscope imaging system 1 of the present invention. When the output terminal 111 on the first body portion 11 is not electrically connected to the input terminal 1314 on the second body portion 13, the image signal generated by the lens assembly 121 is transmitted via the second physical line 122 in the flexible hollow detection tube 12 to the wireless transmitter unit 112 in the first body portion 11. Then, from the wireless transmitter unit 112, the image signal is wirelessly transmitted to the wireless receiver unit 132 in the second body portion 13 and displayed on the display unit 133, allowing the user to view the image at a remote place without being limited to a relatively short fixed signal transmission range as in the case of wire transmission.

The present invention has been described with a preferred embodiment thereof and it is understood that many changes and modifications in the described embodiment can be carried out without departing from the scope and the spirit of the invention that is intended to be limited only by the appended claims.

What is claimed is:

1. A separable-type endoscope imaging system comprising:
a first body portion having an output terminal and a wireless transmitter unit provided thereon;
a flexible hollow detection tube being located at one end of the first body portion, and having a lens assembly provided therein for generating an image signal; and
a second body portion having an input terminal, a wireless receiver unit, and a display unit provided thereon;
whereby when the output terminal on the first body portion is electrically connected to the input terminal on the second body portion, the image signal generated by the lens assembly is transmitted via the output terminal and the input terminal to the display unit; and when the output terminal is not electrically disconnected to the input terminal, the image signal is transmitted from the wireless transmitter unit on the first body portion to the wireless receiver unit on the second body portion for displaying on the display unit.

2. The separable-type endoscope imaging system as claimed in claim 1, wherein the second body portion has a display jack provided thereon for an external display unit to connect thereto; whereby the image signal can be transmitted to an external display unit when the external display unit is connected to the display jack.

3. The separable-type endoscope imaging system as claimed in claim 1, wherein the second body portion has a display jack provided thereon for an external display unit to connect thereto; whereby when an external display unit is connected to display jack, the image signal is transmitted to the display unit on the second body portion and also to the external display unit.

4. The separable-type endoscope imaging system as claimed in claim 1, wherein the first body portion has a first power supply unit provided thereon.

5. The separable-type endoscope imaging system as claimed in claim 4, wherein when the output terminal is electrically connected to the input terminal, the first power supply unit can supply power to the first body portion and the second body portion.

6. The separable-type endoscope imaging system as claimed in claim 1, wherein the second body portion has a second power supply unit provided thereon.

7. The separable-type endoscope imaging system as claimed in claim 6, wherein when the output terminal is electrically connected to the input terminal, the second power supply unit can supply power to the first body portion and the second body portion.

8. The separable-type endoscope imaging system as claimed in claim 1, wherein the flexible hollow detection tube is separably connected to the first body portion.

9. The separable-type endoscope imaging system as claimed in claim 1, wherein the first body portion, the flexible hollow detection tube, and the second body portion are internally provided with a first physical line, a second physical line, and a third physical line, respectively, for wire transmission of the image signal.
